(19) United States
(12) Patent Application Publication
(10) Pub. No.: US 2008/0195155 A1
(43) Pub. Date: Aug. 14, 2008

(54) LOCKING INSTRUMENT FOR IMPLANTABLE FIXATION DEVICE

(76) Inventors: Jeffrey Hoffman, Marquette, MI (US); Gregory Berrevoets, Skandia, MI (US); Maria Norman, Negaunee, MI (US)

Correspondence Address: FITCH EVEN TABIN AND FLANNERY, 120 SOUTH LA SALLE STREET, SUITE 1600, CHICAGO, IL, 60603-3406, US

(21) Appl. No.: 12/029,983

(22) Filed: Feb. 12, 2008

Related U.S. Application Data
(60) Provisional application No. 60/889,368, filed on Feb. 12, 2007.

(51) Int. Cl. A61B 17/70

(52) U.S. Cl. 606/278

(57) ABSTRACT

Instruments are provided for linearly inserting a locking member into a coupling member that secures a rod to a bone or portion of a bone. A moveable drive member of the instrument drives the locking member into engagement with the coupling member, securing the rod therebetween.

### CROSS-REFERENCE TO RELATED APPLICATIONS

This application claims the benefit of U.S. Provisional Application Ser. No. 60/889,368, filed Feb. 12, 2007, and entitled “Locking Instrument for Implantable Fixation Device,” which is hereby fully incorporated by reference as if set forth herein.

### FIELD OF THE INVENTION

The present disclosure relates to an instrument for securing a spinal rod relative to the spine and, more particularly, to an instrument for use in securing a spinal rod to a coupling member that is anchored to a bone.

### BACKGROUND OF THE INVENTION

In a number of surgical procedures, implant devices are utilized to promote the healing and repair of various parts of the human body. In some cases, implant devices secure bones or bone segments relative to each other so that the bones themselves may heal, fuse, or be repositioned. For instance, two or more vertebrae of the spinal column may be linked together by a plate or an elongated rod member in order to prevent relative movement between the vertebrae. Alternatively, an elongated rod may be used to rotate or de-rotate one or more vertebrae relative to at least one other vertebra, such as in treatments for scoliosis, where undesirable torsion of the spine is corrected by “de-rotating” one or more out-of-phase vertebrae to place them in proper rotational alignment with the other vertebrae. In still other cases, implant devices are used to secure a plurality of bones or bone fragments so that soft tissues proximally located to the bones may heal without being disturbed by relative movement of the bones.

Typically, implanting devices that secure bones or bone segments relative to each other involves securing a plurality of coupling members to a plurality of respective bones using anchor members such as bone screws, hooks, or other fixtures. Then, each of the fixtures is secured relative to the others with an additional apparatus, such as a connecting rod. A pedicle screw and rod system is one such example that is commonly used to connect adjacent vertebrae together.

As an example, a patient may require having a number of vertebrae or vertebral fragments secured so that damaged vertebrae may heal, fuse, and/or be repositioned. A number of bone screws may be secured to a plurality of vertebrae or vertebral segments. Each screw may be integrally attached to or threaded through a coupling member, which includes physical structures for coupling a bone screw to a connecting rod. Often, the coupling member includes opposed, upstanding walls to form a yoke within which the connecting rod is retained. Each coupling member may be secured with, and relative to, at least one other coupling member by the spinal rod. A locking member, such as a locking cap, is locked to the coupling member to secure the spinal rod relative to the coupling member.

A number of methods may be used to lock a spinal rod within a coupling member. For instance, many variations of top-loading locking caps have been disclosed. Traditional top-loading locking caps require at least partial rotation of a cap relative to a coupling member in order to loosely secure the cap to the coupling member. Further rotation of the cap provides additional locking force that compresses the rod into the coupling member and locks it into place. Many pedicle screws, for instance, utilize a threaded locking cap that engages threads on the interior or exterior of the yoke so that rotation of the cap relative to the yoke results in linear movement of the locking cap toward the spinal rod. Threading the cap into the coupling member causes an increase in the force securing the spinal rod. When the cap is rotated enough times, a clamping force is provided to secure the rod between the yoke and the locking cap. Other locking devices (such as in U.S. Pat. Nos. 5,084,049 to Asher; 6,565,565 to Yuan; and 6,755,829 to Bono) include locking caps with discrete flanges or slots that may be lowered onto or into a coupling member and then twisted into place with a partial rotation to capture a spinal rod within the coupling member. In such devices, the locking cap can fall out of the coupling member unless the flanges or slots are rotated into contact with corresponding structures on the coupling member.

Alternatively, a novel, axially inserted multi-part locking cap assembly is disclosed in U.S. Utility patent application Ser. No. 11/839,843, which is hereby fully incorporated by reference as if set forth herein, wherein the walls of the coupling member or yoke flex outward to receive a cap and then inward to capture the cap in a snap-lock fit in response to axial, non-rotational movement of the cap relative to the yoke. In one device disclosed in that application, a cap is inserted into the yoke without rotation to a first snap-lock position within the yoke, at least loosely capturing the rod within the yoke. Further axial insertion may lead to one or more additional snap-lock positions, and rotation of at least a portion of the locking cap assembly may be used to provide additional locking force through a camming action between different components of the cap assembly.

Instruments for rotational locking of a locking cap within a yoke have been disclosed (see, for instance U.S. Pat. Publication 2003/0225408). However, such instruments are unsuitable for use with fixation devices having a locking cap that is axially inserted and snap-locked into a coupling member of the fixation device, since the instruments contain clamps that would hinder or prevent flexion of the coupling member to axially receive the locking cap into one or more snap-locking positions. In addition, prior art tools are not designed to provide an axial driving force sufficient to overcome resistance from the coupling member in order to drive the locking cap into locking engagement with the coupling member. In prior art devices, simple axial movement of a locking cap merely positions the locking cap in the coupling member, and significant resistance is not encountered until rotation of the locking cap.

Therefore, there remains a need for an improved insertion instrument for use with implant devices having a locking cap that is axially inserted to achieve one or more locking positions.

### SUMMARY OF THE INVENTION

The present invention is related to devices and methods to facilitate securing an implantable structure, such as a spinal rod, to a fixation device, such as a pedicle screw or vertebral hook.

In one aspect, a locking cap insertion instrument is provided for engaging a coupling member or yoke of a fixation device, and driving the locking cap into locking engagement with the yoke to secure a spinal rod therein. The insertion instrument contains a stationary member and a moveable member. The stationary member has a seat for receiving the yoke mounted to the vertebra (such as disclosed in pending application 60/825,366) and the moveable member is configured to linearly or axially advance the locking cap toward the seat, and thus into engagement with the yoke.

The locking cap insertion instrument may have an elongate body member in the form of a handle. The fixed member may extend axially from the body member, positioning the seat at a fixed distance from the body member. The seat has an opening in order to receive the yoke of the fixation device when the yoke is mounted to a bone, such as a vertebra. The opening of the seat is sized such that when the yoke is sealed therein, engagement between the seat and the yoke restricts movement of the seat away from the bone. The instrument may also contain a moveable drive member extending axially from the elongate body toward the seat of the fixed member. The moveable drive member is linearly or axially moveable relative to both the body member and the fixed member toward the seat of the fixed member. The distal end or tip of the moveable drive member is configured to releasably hold the locking cap for insertion into the yoke. An actuating member may be provided in order to effect axial movement of the moveable drive member. In one embodiment, the locking cap insertion instrument contains an actuator lever operably connected to the moveable member. Moving the actuator lever relative to the body member axially advances the moveable member in order to advance the cap assembly and spinal rod into a yoke.

The fixation device manipulated with the insertion tool described herein may be, for instance, a pedicle screw assembly including a coupling member (such as a yoke) that is anchored to bone and designed to receive an elongate spinal rod, and a locking cap that is axially engaged to the coupling member.

In one form, the spinal rod is placed within the yoke, and then captured by axial insertion of the locking cap over the rod to secure the locking cap and rod to the yoke. To simplify assembly and operation, it is preferred that the locking cap insertion instrument releasably engages the cap and provides an axial force to push the cap against the spinal rod, forcing both the rod and cap into an open end of the yoke. The force exerted by the insertion instrument causes contact between the locking cap and the yoke such that the locking cap is secured to the yoke and closes the open end of the yoke, with the spinal rod disposed therebetween.

More particularly, it is preferred that the insertion instrument directs the locking cap and the spinal rod along an axial path along a central axis of the yoke. To achieve this, it is further preferred that the yoke have a closed end opposite the open end, with the closed end configured to be received in the seat of the insertion tool. The distal end or tip of the moveable drive member faces the seat, so that axial movement of the drive member moves the tip end, and any locking cap thereon, toward and away from the open end of the yoke and along an axis of the yoke. This configuration provides a generally pre-determined orientation between the moveable drive member and the yoke when the yoke is received in the seat of the insertion tool.

During operation, axial movement of the drive member pushes the locking cap and spinal rod into the open end of the yoke. More specifically, the drive member engages and pushes against the locking cap, which in turn causes the lower surface of the locking cap to abut and thereafter advance against the spinal rod such that the locking cap and spinal rod are both advanced into the open end of the yoke. Though it is preferred that the locking cap does not rotate during advancement, the cap may be rotated to further lock or capture the rod within the yoke walls after axial insertion into the yoke. In this manner, the locking cap is captured in the yoke upon axial insertion so that the insertion tool may be withdrawn without risk of the cap becoming disconnected from the yoke. Once the insertion tool is withdrawn from the locking cap that is captured in the yoke, the locking cap will not exit the yoke. The cap may be designed so that linear insertion fully locks the spinal rod within the yoke. The locking cap may alternatively be designed to be rotated or otherwise manipulated for final locking using a separate instrument. The insertion instrument may be equipped with a rotatable drive member so that linear advancement and final locking are accomplished with a single tool. In order to provide for rotational locking, the locking cap may include a recess in which a mating end portion of a locking tool may be received.

### BRIEF DESCRIPTION OF THE DRAWINGS

FIG. 1 is a side elevation view of an insertion instrument in a retracted position.

FIG. 2 is a side elevation view of the insertion instrument engaged to a yoke and positioned for axial insertion of a locking cap.

FIG. 3 is a side elevation view of the insertion instrument with the locking cap inserted into the yoke.

FIG. 4 is an exploded perspective view of the insertion instrument.

FIG. 5 is a cross-sectional elevation view of the insertion instrument in the position shown in FIG. 1 and showing the actuator member and drive member in the retracted position.

FIG. 6 is a cross-sectional view of the insertion instrument in the position shown in FIG. 3 and showing the actuator member and the drive member in the extended positions.

FIG. 7 is a perspective view of a seat of the insertion tool of FIG. 1.

FIG. 8 is a perspective view of the seat of the insertion instrument engaging the yoke.

FIG. 9 is a side elevation view of a drive member positioning the locking cap for axial insertion into the yoke.

FIG. 10 is a side elevation view similar to that of FIG. 9, but showing the drive member inserting the locking cap axially into a first position within a yoke.

FIG. 11 is a side elevation view similar to that of FIGS. 9 and 10 but showing the drive member inserting the locking cap axially into a second position within the yoke.

### DETAILED DESCRIPTION OF THE PREFERRED EMBODIMENTS

In one form, an insertion instrument is provided having an elongate profile, with a proximal end configured for use by a surgeon as a handle and a distal end configured to operably engage one or more components of a fixation device. The insertion instrument contains a rod-shaped moveable drive member configured to move linearly, or axially, relative to the handle. A fixed member comprising an elongate shaft and a seat located at the distal end of the instrument is configured to engage a yoke and aligns the yoke with the axial pathway of the moveable drive member. The insertion instrument also contains an actuator operably connected to the moveable member for controlling axial movement of the moveable member relative to the instrument. A locking cap can be placed in the end of the moveable member and, using the actuator, moved into engagement with the yoke to clamp a rod or other spinal implant therebetween.

In one form, the insertion instrument disclosed herein is suitable for use with the spinal rod anchoring system described in the commonly assigned co-pending Application No. 60/825,366, the specification of which is incorporated herein by reference in its entirety as if reproduced herein. Such a system contains a locking cap that is axially inserted into an open end of an essentially cylindrical yoke, in order to capture a spinal rod within the yoke without rotation of the cap. Preferably, axially advancing the locking cap at least partially into the yoke causes a snap-lock fit between the cap and yoke, wherein the walls of the yoke deflect away from the locking cap as the cap is inserted axially into the open end of the yoke, and then return to their original position once the cap is disposed at least partially within the yoke. Once the rod has been captured in the yoke, the locking cap (or a portion of a multi-part locking cap assembly) may be rotated in order to provide further compression that further restricts movement of the spinal rod, locking it in place within the yoke.

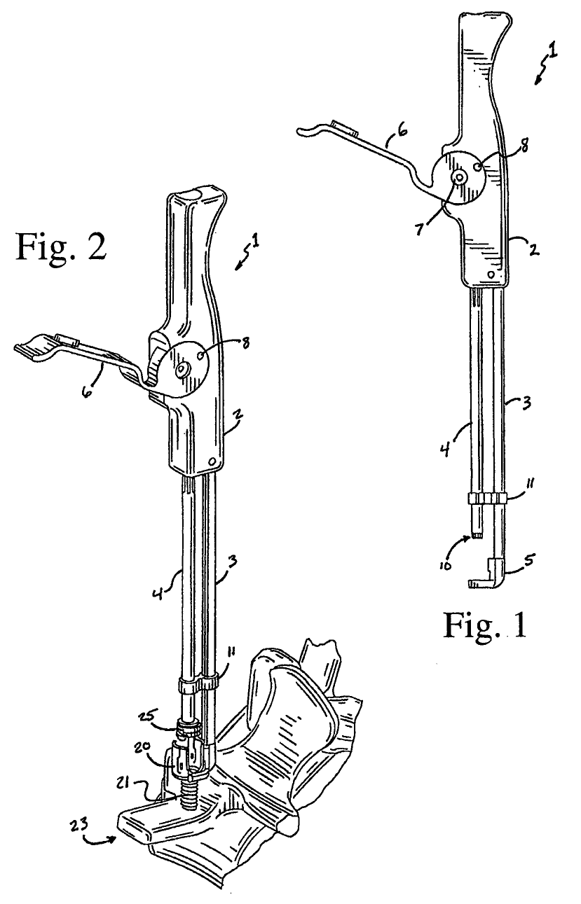

Referring to FIG. 1, one particular embodiment of an insertion instrument for securing a locking cap to a coupling device with a rod therebetween is illustrated. The surgical instrument is designed to engage a yoke connected to a movable or fixed anchor that is implanted in a vertebra, and to push a locking cap into contact with the yoke in order to at least partially secure the locking cap to the yoke. The instrument 1 comprises an elongate body 2 that may be gripped by the surgeon during use. In the embodiment shown, the elongate body 2 forms a contoured handle designed to match generally the contours of a user's palm as the palm grips the handle. Two rod-shaped members extend from the elongate body 2, including a fixed member 3 having at one end a seat 5 for receiving the yoke of a pedicle screw or other fixation device, and a drive member 4 extending from the body in a direction parallel to the fixed member.

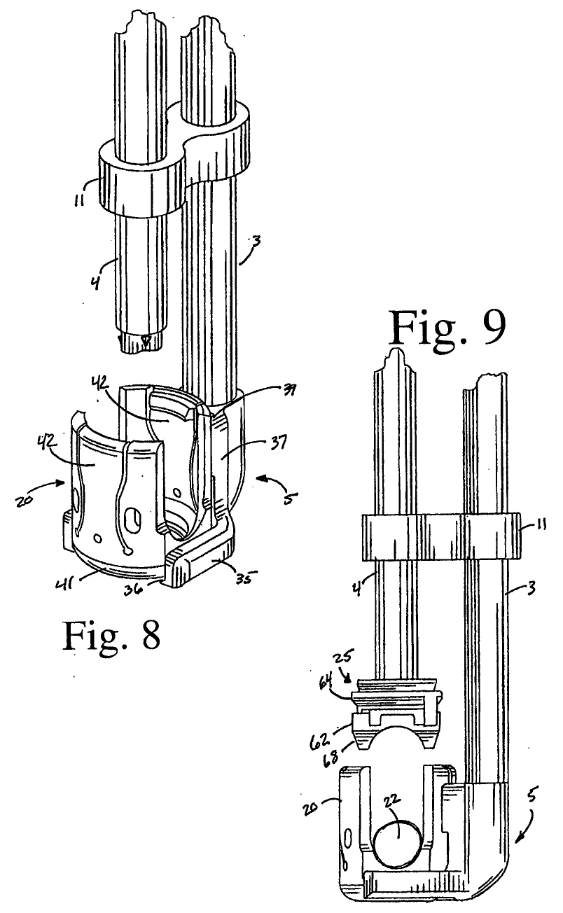

The drive member tip 10 is configured to receive and releasably engage a locking cap and hold the locking cap until the locking cap is secured to the yoke. As shown in FIG. 8, the tip 10 comprises a lobed configuration designed to fit within a complementary lobed recess in the top of a locking cap. To provide a tight, friction fit with the locking cap, the drive member tip 10 may also includes at least one, and preferably two, wedge inserts located between adjacent lobes. The inserts permit the profiled tip 10 of the drive member 4 to securely hold the cap in a friction-tight fit and generally provide an improved ability to secure the cap to the drive member 4 over a more traditional, tapered press-fit design of the drive member end. In one form, the wedge inserts are thermoplastic resin inserts and have a shape to conform to the region between two adjacent lobes on the drive member tip 10. Suitable inserts may be provided by Nemcomed, Ltd. (Hicksville, Ohio); however, other inserts and/or materials may also be used with the drive member 4. It will be appreciated that any number and shape of inserts may be provided to correspond to the profile on the drive member tip 10.

The drive member 4 is slidably disposed within the elongate body 2 so that it may be axially driven toward the seat 5. The drive member 4 and fixed member 3 are bridged by a guide device or collar 11 that is secured to the fixed member 3 and provides a guide for the drive member 4 at a fixed distance perpendicular to the long axis of the fixed member to stabilize the drive member 4 as it moves axially and parallel to the fixed member 3.

With continuing reference to FIG. 1, an actuator lever 6 is pivotally secured to the elongate body 2 by a bolt 7. The actuator lever 6 is biased away from the body, as will be described below. A user may pull the lever 6 toward the elongate body 2, causing the lever 6 to pivot about the bolt 7, which in turn causes axial movement of the drive member 4, which is linked to the actuator lever 6 by a pin 8. The base of the actuator lever 6 comprises a drive wheel 26 that is disposed partially within a corresponding recess of the body 2 (as shown in FIG. 4). In the illustrated embodiment, the drive wheel 26 is formed integrally with the actuator lever 6. Alternatively, the base of the lever may comprise other shapes, and may be disposed fully on the exterior or interior of the body 2.

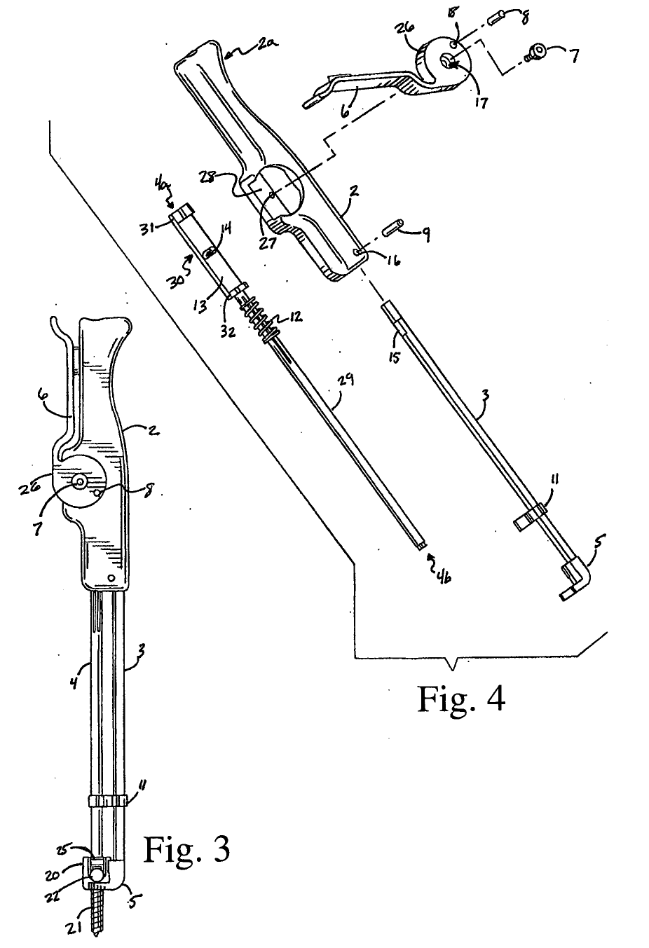

The instrument 1 is used with a yoke 20 of a fixation device and a locking cap 25 designed to lock a spinal rod 22 within the yoke 20, as illustrated in FIGS. 2 and 3. The yoke comprises upstanding side walls and a channel for receiving the spinal rod. Depending from the yoke 20 is a bone screw 21 that is anchored to a vertebra 23 of a patient. The yoke 20 is received in the seat 5 of the fixed member 3 of the instrument 1. The seat 5 aligns the yoke 20 with the drive member 4 for insertion of the locking cap 25, and prevents the instrument from moving away from the yoke 20 during insertion. Advantageously, the seat 5 may be configured to loosely receive the yoke 20 in order to facilitate rapid engagement and disengagement of the instrument 1 to the yoke 20.

Movement of the actuator lever 6 toward the elongate body 2 causes drive member 4 to insert the locking cap 25 into the yoke 20, capturing the spinal rod 22 between the locking cap 25 and the yoke 20, as illustrated in FIG. 3. Movement of the actuator lever 6 toward the body 2 of the instrument causes a pin 8, which is attached to the drive wheel 26 of the lever 6, to pivot about the bolt 7 that fixes the lever to the body. Since the drive pin 8 engages the drive member 4 as well as the drive wheel 26 of the actuator lever 6, rotational movement of the wheel 26 and drive pin 8 causes axial movement of the drive member 4 relative to the body 2. When the lever 6 is fully compressed toward the body 2, drive member 4 preferably fully axially inserts the locking cap 25 into the yoke 20.

Turning now to more of the details of construction of the insertion instrument 1, as shown in FIG. 4, the elongated body member 2 has a proximal end 2a and a distal end 2b. The proximal end 2a of the body is configured to be gripped by a surgeon, while the distal end 2b receives structures operable to move the locking cap toward the yoke of the fixation device. The distal end 2b of the body member 2 is configured to receive the fixed member 3. The fixed member 3 may alternatively be formed integrally with the body member 2. In the embodiment shown in FIG. 4, the fixed member 3 is a rod-shaped component that is partially inserted into the elongate body member 2 parallel to the long axis of the elongate body member 2. In the illustrated embodiment, the fixed member 3 has a flat recess 15 along one side that aligns with an opening 16 in the body when the fixed member 3 is inserted into the body. A pin 9 is inserted through the opening 16 in the body in a direction transverse to the axis of insertion of the fixed member 3, in order to provide a press fit between the pin 9 and the recess 15 of the fixed member 3 to secure the fixed member 3 in place and prevent rotation of the fixed member 3 relative to the body 2.

The drive member 4 has a proximal end 4a positioned in the body or handle and distal end 4b that moves the locking cap. In the illustrated embodiment, the drive member 4 comprises a relatively narrow, elongate cylindrical shaft 29 at the distal end 4b and a wider stop portion 30. The stop portion 30 is essentially cylindrical, with a broad recessed flat 13 on one side. This recessed flat 13 has a length in the axial direction greater than the diameter of the drive wheel 26 of the actuator lever 6. The recessed flat 13 contains an elongated slot 14 oriented transverse to the axis of the drive member 4. The slot 14 opens to at least one side of the stop portion 30. In the illustrated embodiment, the slot 14 traverses the width of the stop portion 30 from the face of the recessed flat 13 to an opposing side of the stop portion, opening at two opposite sides of the stop portion. A compression spring 12 is disposed concentrically outside of the end of the cylindrical shaft 29 of the drive member so that it abuts a proximal end 32 of the wider stop portion 30.

When the components shown in FIG. 4 are assembled, the drive member 4 is disposed partially in the body member 2, with the narrow shaft 29 of the drive member 4 extending from the distal end of the body member 2. The stop portion 30 of the drive member 4 is disposed within the body member 2, with the flat recess 13 of the stop portion 30 facing outward through a large opening 28 in the body member 2. The drive wheel 26 of the actuator lever 6 is received in the large opening 28 of the body member 2 and held in place by a bolt 7 that is disposed through a central opening 17 of the drive wheel 26 of the lever 6 and then is threaded into a threaded pivot opening 27 on the body 2. When held in place by the bolt 7, the drive wheel 26 of the actuator lever 6 is positioned so that it is adjacent to the flat recess 13 in the stop portion 30 of the drive member 4. Since the flat recess 13 of the drive member has a length greater than the diameter of the wheel 26 of the actuator lever 6, the drive member 4 is able to move axially adjacent to the drive wheel 26 of the lever 6 without the ends 31 and 32 of the stop portion 30 abutting against the drive wheel 26 of the lever 6. An opening 18 located radially outward from the central opening 17 of the drive wheel 26 receives the drive pin 8 that abuts the drive wheel 26 and the drive member 4, linking rotation of the wheel and linear travel of the drive member. When inserted, a leading end of the drive pin 8 extends through the opening 18 in the drive wheel 26 and is received in the elongated slot 14 of the drive member 4. The drive pin 8 may be fixed to the wheel 17 by press fitting, soldering, threads, or other means of attachment, and may alternatively be formed integrally with the wheel.

Due to the abutment of the pin 8 and the drive member 4, movement of the actuator lever 6 is able to effect movement of the drive shaft 4. Pivoting movement of the lever 6 rotates the wheel 26, causing angular displacement of the driving pin 8 located in the opening 18. This angular displacement of the pin 8 pushes the pin against the interior of the slot 14 in the drive member 4, axially moving the entire drive member 4. The drive member 4 is confined within an elongate channel inside the body member 2 so that its movement is limited to an axial direction.

When the drive member 4 is disposed in the body member 2, the distal portion of shaft 29 of the drive member extends from the body member distal end 2b, parallel to the fixed member 3. The elongated shaft 29 of the drive member 4 also passes through a guide opening in the collar 11 that engages the fixed member 3. Preferably, the collar is secured to the fixed member in a manner that limits or prevents movement of the collar. The collar 11 bridges the distance separating the drive member 4 and fixed member 3, holding the drive member and fixed member parallel and preventing them from splaying. The collar 11 also provides additional rigidity to the fixed member 3 and drive member 4, reducing the risk of bending or breaking of one or both members during operation.

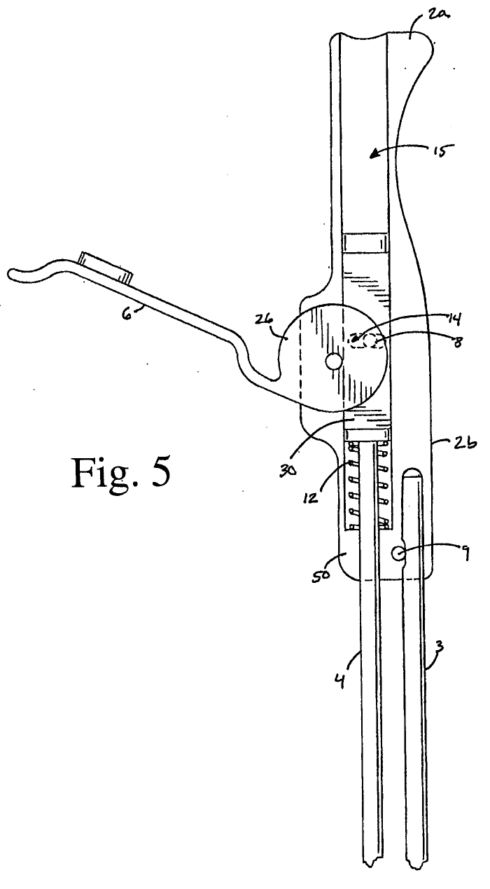

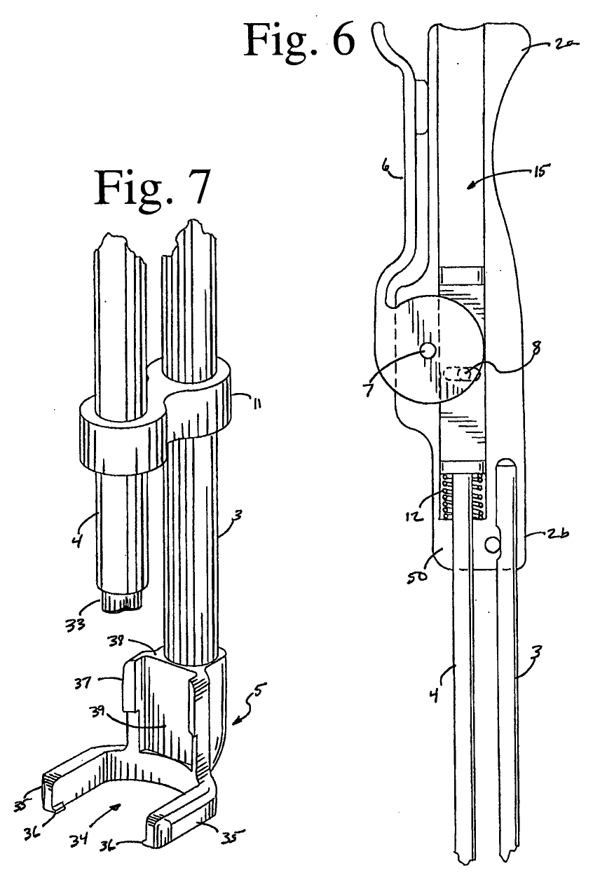

FIGS. 5 and 6 illustrate cross-sectional views of the insertion tool 1 during operation. FIG. 5 shows the actuator lever 6 in a first open position. The wheel 26 forming the base of the actuator lever 6 is fixed to the elongate body 2 of the tool by a bolt 7 disposed in the center of the wheel 26. The drive member 4 is linked to the wheel 26 by a drive pin 8 located radially outward from the rotational center of the wheel 26, with the driving pin being capable of rotating with respect to at least one of the wheel 26 and the drive member 4. The drive member 4 is disposed in an elongate channel 15 running axially through the body member 2. The channel 15 is open at both the proximal end 2a and distal end 2b of the body member 2, with a circumferential lip 50 formed around the opening in the distal end to create a narrower opening at the distal end of the channel 15 than at the proximal end. The proximal opening of the channel 15 is of greater diameter than the widest portion of the drive member 4, so that the drive member may be inserted into the channel through the proximal end. The distal opening of the channel 15 has a diameter greater than the diameter of the shaft portion 29 of the drive member 4 but less than the diameter of the stop portion 30, so that the narrower shaft portion 29 may extend through the opening in the channel 15 and extend distally from the body member 2, but without the full drive member exiting completely through the distal opening in the channel 15. A compression spring 12 is disposed between the distal end of the stop member 30 and the circumferential lip 50 around the distal opening of the channel 15 to bias the drive member toward the proximal end of the body 2. The force supplied by the compression spring 12 pushes the drive member 4 axially along the channel 15 toward the proximal end 2a of the body 2. This linear force applied to the drive member 4 causes rotational movement of the wheel 26 at the base of the actuator lever 6, biasing the lever outward away from the body member 2. The drive member 4 is prevented from exiting the proximal opening of the channel 15 due to abutment with the drive pin 8, which is disposed in the wheel 26, which is in turn attached to the body member 2 by the bolt 7.

In FIG. 6, the actuator lever 6 has been shifted toward the body member 2, causing rotation of the wheel 26 at the base of the lever 6 and angular displacement of the drive pin 8 disposed in the wheel 26. The change in position of the pin 8 forces the drive member 4 to move axially in the distal direction, causing the compression spring 12 to be compressed between the stop member 30 and the circumferential lip 50 of the distal opening of the channel 15. Thus, as the surgeon squeezes the actuator lever 6 toward the body 2, the drive member 4 is driven into an extended position in the distal direction, away from the elongate body member 2. When the actuator handle 6 is released by the surgeon, the compression spring 12 causes the drive member 4 to retract and pushes the actuator lever 6 outward away from the body back into the open position as depicted in FIG. 5.

Biasing the moveable drive member 4 away from the seat 5 of the fixed member 3 advantageously holds the drive member in a retracted position at a distance from the seat when the drive member is idle, allowing the yoke to be situated in the seat 5 without interference from other portions of the instrument. Not only does this biasing of the drive member prevent obstruction of the seat 5 of the fixed member 3, it also provides for automatic withdrawal of the drive member from the insertion site after the locking cap 25 has been driven into locking engagement with the yoke 20. When the surgeon releases the actuator lever 6, so that axial force is no longer applied to the drive member 4, the drive member 4 will automatically retreat toward the proximal end of the instrument. Therefore, as long as the locking force between the locking cap and yoke is greater than the force holding the locking cap to the drive member, release of the actuator member causes the drive member to automatically release from the inserted locking cap and withdraw from the insertion site, preparing the instrument for disengagement from the fixation device. The described automatic release feature, combined with a one-piece seat without clamping means, allows the instrument to be rapidly engaged and disengaged to a series of fixation devices and operated with one hand by squeezing and releasing a single actuator.

Turning now to details of the seat of the insertion tool, shown in FIGS. 7 and 8, the seat 5 is configured to receive the yoke of the fixation device and is held at a predetermined position from the body 2 at the distal end of the fixed member 3 of the insertion tool. The moveable drive member 4 is moveable axially toward and away from the seat 5. The distal tip 33 of the drive member 4 is configured to receive and hold the locking cap, as discussed above. In the embodiment shown, the seat 5 comprises two outwardly protruding arms 35 each having a small flange 36 protruding toward the other arm. The arms 35 form an open space 34 into which the yoke is received from a direction perpendicular to the axis of the drive member 4. Flanges 37 on the upright portion 38 of the seat 5 are also configured to receive the exterior of the yoke. The flanges 37 form a vertical recess 39 in the seat, allowing a flexible portion of the yoke to flex outward into the recess during axial insertion of the locking cap.

In FIG. 8, the yoke 20, is received in the seat 5 of the insertion tool. Advantageously, the seat of the fixed member may be predominately open so as not to interfere with flexion or other movement of moveable portions of the yoke. In one form, the yoke has a base portion 41 with deflectable side wall portions 42 that deflect to provide a snap-fit with a linearly-inserted locking cap. By engaging the base portion 41 of the yoke and avoiding the use of clamp members or other structures that mount to and tightly engage the upstanding walls of the yoke, the seat 5 avoids obstruction of flexion that may be required to lock the cap within the yoke.

When the locking cap is designed to lock in a snap-fit connection with the yoke, the length of the drive rod, and surface features of the cap and yoke may be selected to provide one or more stages of axial locking insertion. For instance, advancing the cap to a first fixed distance may provide a first stage of snap-fit locking between a lower part of the cap and the yoke, while advancing to a further second fixed distance provides a second stage of snap-fit locking between an upper part of the cap and the yoke.

In the embodiment shown, the yoke 20 comprises a yoke having opposed upstanding side walls 42. A bone screw 21 depends from the yoke 20 between the arms of the seat 35, and is anchored to a vertebra (as shown in FIG. 2). The yoke 20 is held loosely by the arms 35 of the seat, with flanges 36 positioned under the base member 41 of the yoke 20 to prevent the yoke from moving through the arms 35. A recess 39 in the seat is aligned with the flexible portion 42 on one side of the yoke, so that the seat does not obstruct outward camming of the flexible portion 42 as a locking cap is inserted linearly into the yoke. The seat 5 is open on one side to allow the arms 35 to engage and disengage the yoke 20. The upright flanges 37 on the seat are configured to match the contour of the exterior of the side walls 42 of the yoke. The seat is configured so that the central axis of the yoke 20 is in line with the drive member 4. The collar 11 bridging the fixed member 3 and drive member 4 ensures that the drive member 4 maintains alignment with the central axis of the yoke 20 while the drive member moves axially toward the yoke.

The drive member 4 operates to capture a spinal rod and locking cap in the yoke of the fixation device, as shown in FIGS. 9-11. Referring to FIG. 9, a locking cap in the form of a locking cap assembly 25 is shown releasably engaged to the tip of the drive member 4. The yoke 20 is located directly below the locking cap assembly and is received in a seat 5 in a fixed position so that the drive member 4 and yoke 20 are coaxial. A spinal rod 22 is seated within an open channel of the yoke 20. The bottom of the locking cap contains a first set of inwardly-tapered surfaces 68 configured to guide the locking cap into the yoke 20 during axial insertion. As the locking cap assembly 25 is driven toward the yoke 20, the tapered surfaces 68 of the locking cap act as a wedge and slide between the side walls 42 of the yoke 20. The insertion of the locking cap assembly 25 into the yoke 20 is achieved via flexible or resilient portions of the side walls 42 of the yoke. The side walls 42 resiliently or elastically flex as the locking cap assembly 25 is inserted linearly from the top of the yoke 20 in order to permit the receipt of the locking cap into an internal space between the side walls 42 of the yoke. The resilient portions of the side walls 42 are sufficiently flexible to allow flexion or camming thereof in a direction transverse to the insertion direction, but are also sufficiently stiff to return to their original position and retain the locking cap 25 in a locked position once the locking cap has been inserted to one or more predetermined locking positions within the yoke.

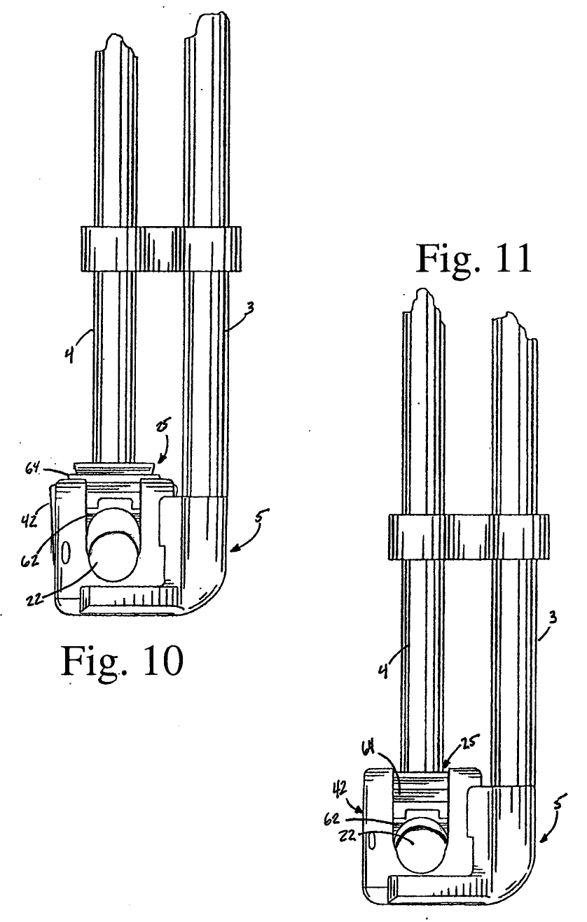

When the lower portion of the cap assembly 62 clears the top of the side walls 42 and enters the internal space of the yoke, a first locking position is reached and the resilient portions of the side walls return to their original position, capturing the lower portion of the cap 62 within the yoke 20, as illustrated by FIG. 10. In this first locking position, a relatively large gap is formed between the locking cap 25 and the spinal rod 22. Although the spinal rod is prevented from exiting through the top of the yoke 20 by the locking cap 25, there is sufficient space within the yoke to permit shifting of the spinal rod both along the yoke axis and transverse to the yoke axis in order to permit the surgeon to make relatively large adjustments to the position of the spinal rod such as may be necessary to accommodate spines with extensive curvature thereto.

Further linear advancement of the locking cap 25 achieves a second locking position, as shown in FIG. 11. Once the surgeon has positioned the spinal rod 22 near its final orientation and position, additional axial force is supplied to the drive member 4 to drive the cap 25 further into the yoke 20, applying a compression force to the spinal rod 22 and reducing the space in which the rod may move. Similar to entry into the first locking position, resilient portions of the side walls flex outward to receive the upper portion of the cap 64. Once the upper portion 64 has cleared the top of the side walls 42, the resilient portions shift inwardly to capture the entire cap 25.

Advantageously, the instrument described herein requires relatively few parts, and especially few moving parts, to provide an instrument that is easily assembled, disassembled, and cleaned. Sterilization of the linear insertion instrument is simplified by providing an elongate shape without moveable parts at the distal end. Although the drive member inserts a locking cap into a coupling member located in the surgical site, the actuating mechanism causing movement of the drive member is contained entirely on the proximal body portion of the instrument, thereby avoiding crevices, seams, and other formations on the distal end of the instrument capable of harboring blood, tissue, and other foreign matter.

While there have been illustrated and described particular embodiments of the present invention, it will be appreciated that numerous changes and modifications will occur to those skilled in the art, and it is intended in the appended claims to cover all those changes and modifications which fall within the true spirit and scope of the present invention.

What is claimed is:

1. A surgical instrument for securing a locking cap to a coupling member, the surgical instrument comprising:
an elongate body;
a fixed member extending from the body having a seat configured for receiving the coupling member; and
a drive member having a tip configured for releasably engaging the locking cap, the drive member being linearly moveable relative to the fixed member to advance the locking cap into the coupling member.

2. The instrument of claim 1, wherein the tip of the drive member has a friction coating for frictionally engaging a recess of the locking cap.

3. The instrument of claim 1, wherein the drive member includes a biasing device for biasing the drive member away from the seat.

4. The instrument of claim 1, wherein the coupling member is received into the seat from a direction perpendicular to the linear movement of the drive member.

5. The instrument of claim 1, further comprising an actuator operable to linearly move the drive member relative to the fixed member.

6. The instrument of claim 5, wherein the actuator comprises a lever pivotally connected to the body.

7. The instrument of claim 6, wherein the lever and the drive member have connecting means therebetween, such that pivoting of the lever member causes linear travel of the drive member.

8. The instrument of claim 5 wherein the drive member and fixed member are elongate members that generally extend parallel to each other.

9. The instrument of claim 1, wherein the fixed member has a guide member fixed thereto configured to guide the drive member for linear travel.

10. A method for securing a locking member to a coupling member having a base portion and upstanding wall portions, the method comprising:
engaging the coupling member along a base portion thereof with a seat portion of a tool;
linearly advancing the locking member with a linearly moving drive portion of the tool with an actuating force applied to the drive portion;
using the engagement between the coupling member with the seat portion of the tool to maintain the linear movement of the locking member in the advancing direction as force is applied to the drive portion; and
camming the upstanding wall portions of the coupling member outward as the locking member is advanced into the coupling member.

11. The method of claim 10, wherein the actuating force is applied by pivoting an actuating member relative to the fixed portion and drive portion of the tool.

12. The method of claim 10, wherein the coupling member is received in a side opening of the seat so that a base portion of the coupling member engages the seat and an anchor member depending from the base portion extends through the opening, with the linear movement of the drive portion guided along a path perpendicular to the opening in the seat portion.

13. A vertebral implant system comprising:
a spinal rod;
a coupling member having a base portion, upstanding side walls extending from a first side of the base portion, and an anchor member extending from a second side of the base portion and anchored to a vertebra;
a locking cap configured for linear insertion into the coupling member;
an insertion tool for inserting the cap into the yoke, the insertion tool having a handle and an actuator moveable with respect to the handle;
a fixed member extending from the handle of the insertion tool, the fixed member having outwardly extending arms that engage the coupling member base from the second side;
a drive member extending from the handle of the insertion tool and moveable with respect thereto by movement of the actuator, the drive member having a portion configured to receive the locking cap.

14. The system of claim 13, wherein the drive member moves toward the first side of the coupling member base portion when the fixed member is engaged with the second side of the coupling member base portion.

15. The system of claim 13, wherein the side walls of the coupling member have flexible portions that flex outward as the locking cap is linearly inserted between the side walls, and the fixed member of the instrument further engages at least one side wall, the fixed member having a recess into which the flexible portion of the at least one side wall flexes.

16. The system of claim 13, wherein the actuator and drive member are connected by a pin.
